(19) United States
(12) Patent Application Publication
(10) Pub. No.: US 2015/0051431 A1
(43) Pub. Date: Feb. 19, 2015

(54) METHODS AND SYSTEMS FOR PRODUCING GASOLINE

(71) Applicant: UOP LLC, DES PLAINES, IL (US)

(72) Inventors: Bryan K. Glover, Algonquin, IL (US); Danielle Schindlbeck, Arlington Heights, IL (US); Mark R. Little, Mount Prospect, IL (US); David James Shecterle, Arlington Heights, IL (US)

(73) Assignee: UOP LLC, DES PLAINES, IL (US)

(21) Appl. No.: 13/968,135

(22) Filed: Aug. 15, 2013

(51) Int. Cl. C07C 5/27

(52) U.S. Cl. CPC C07C 5/2729
USPC 585/671; 422/187

(57) ABSTRACT

Methods and systems for producing gasoline are disclosed. In one exemplary embodiment, a method for producing gasoline includes the steps of isomerizing a first stream comprising normal C₆ hydrocarbons to produce a second stream comprising first and second branched C₆ hydrocarbons and deisohexanizing the second stream to produce a third stream comprising the first and second branched C₆ hydrocarbons wherein the first branched hydrocarbons and the second branched hydrocarbons are present in a first proportion, and a fourth stream comprising the first second branched hydrocarbons wherein the first branched and the second branched hydrocarbons are present in a second proportion. The first proportion has a relative percentage of first branched hydrocarbons that is greater than a relative percentage of first branched hydrocarbons in the second proportion.

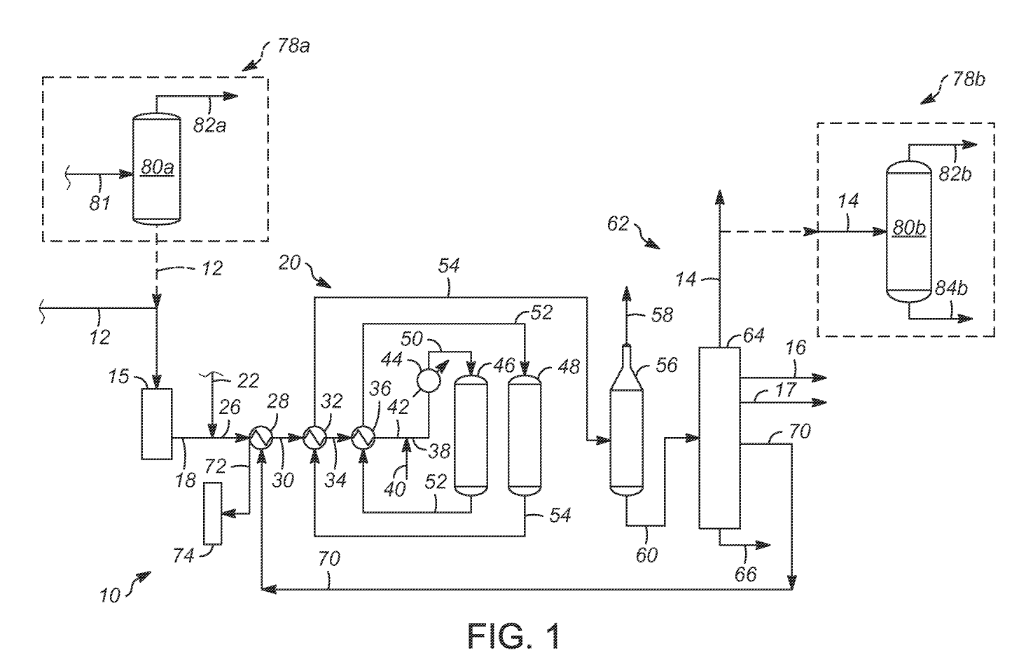

### TECHNICAL FIELD

The present disclosure generally relates to methods and systems for producing gasoline. More particularly, the present disclosure relates to methods and systems for isomerizing and deisohexanizing C₆ hydrocarbons in the production of multiple grades of high-octane gasoline.

### BACKGROUND

Processes for the isomerization of paraffins into more highly branched paraffins are widely practiced. Particularly important commercial isomerization processes are used to increase the branching, and thus the octane value of refinery streams containing paraffins of 4 to 8, especially 5 and 6, carbon atoms. The isomerate is typically blended with a refinery reformer effluent to provide a blended gasoline mixture having a desired research octane number (RON).

The isomerization process proceeds toward a thermodynamic equilibrium. Hence, the isomerate will still contain normal paraffins that have low octane ratings and thus detract from the octane rating of the isomerate. Provided that adequate high octane blending streams (for example, having an RON of about 90 or greater) such as alkylate and reformer effluent (reformate) are available and that gasolines of lower octane ratings, such as 85 and 87 RON, are in demand, the presence of these normal paraffins in the isomerate has been tolerated.

However, when blending certain types of high-octane gasolines, it is often difficult to obtain the gasoline octane that is required (often an RON of 95 or higher) while maintaining aromatics content of the gasoline below 35%. Often, imported materials such as methyl tertiary-butyl ether (MTBE), ethyl tertiary-butyl ether (ETBE), or ethanol are required to achieve high octane in finished gasoline. This is especially the case when multiple octane grades are required because blends composed primarily of reformate and isomerate offer limited flexibility for blending multiple octane grades.

Accordingly, it is desirable to provide methods and systems for producing gasoline at multiple octane grades, and in particular at multiple high octane grades, such as at RONs of about 90 or greater. It is further desirable to provide such methods and systems that do not require the use of imported octane-enhancing materials. Furthermore, other desirable features and characteristics of the present disclosure will become apparent from the subsequent detailed description and the appended claims, taken in conjunction with the accompanying drawings and this background of the disclosure.

### BRIEF SUMMARY

Methods and systems for producing gasoline are disclosed. In one exemplary embodiment, a method for producing gasoline includes the steps of isomerizing a first stream comprising normal C₆ hydrocarbons to produce a second stream comprising first and second branched C₆ hydrocarbons and deisohexanizing the second stream to produce a third stream comprising the first and second branched C₆ hydrocarbons wherein the first branched hydrocarbons and the second branched hydrocarbons are present in a first proportion, and a fourth stream comprising the first second branched hydrocarbons wherein the first branched and the second branched hydrocarbons are present in a second proportion. The first proportion has a relative percentage of first branched hydrocarbons that is greater than a relative percentage of first branched hydrocarbons in the second proportion.

In another exemplary embodiment, a system for producing gasoline includes an isomerization unit configured to isomerize normal C₆ hydrocarbons into first and second branched C₆ hydrocarbons. The system further includes a deisohexanizing unit, fluidly coupled with the isomerization unit, and configured to separate the first branched C₆ hydrocarbons from the second branched C₆ hydrocarbons. The deisohexanizing unit is further configured to produce a first product stream comprising the first branched and the second branched hydrocarbons in a first proportion, and a second product stream comprising the first branched and the second branched hydrocarbons in a second proportion. The first proportion has a relative percentage of first branched hydrocarbons that is greater than a relative percentage of first branched hydrocarbons in the second proportion.

### BRIEF DESCRIPTION OF THE DRAWING

The gasoline producing systems and associated methods will hereinafter be described in conjunction with the FIGURE, which illustrates a method implemented on a gasoline producing system in accordance with various embodiments of the present disclosure.

### DETAILED DESCRIPTION

The following detailed description is merely exemplary in nature and is not intended to limit the disclosure or the application and uses of the disclosed embodiments. All of the embodiments and implementations of the gasoline producing systems and associated methods described herein are exemplary embodiments provided to enable persons skilled in the art to make or use the same and not to limit their scope, which is defined by the claims. Furthermore, there is no intention to be bound by any expressed or implied theory presented in the preceding technical field, background, brief summary, or the following detailed description.

Embodiments of the present disclosure are generally directed to continuous catalytic processes, and catalytic reactors implementing such processes, used in the refining of crude oil to produce gasoline. The processes isomerize hydrocarbon feeds into higher octane, branched molecules. For example, a hydrocarbon feed such as light naphtha, which typically includes C₄-C₇ paraffins and C₅-C₇ cyclic hydrocarbons, and often primarily includes C₅ and C₆ paraffins, may be isomerized into higher-octane, branched C₅/C₆ molecules. The processes typically use catalytic reactors with high activity chlorinated alumina-type platinum, S—Zr-type, and zeolitic-type catalysts. A single pass of feedstock with an octane rating (RON) of about 50 to about 60 through such a reactor typically produces an end product rated at about 76 to about 82. To obtain a higher octane rating, the feedstock may be subsequently passed through a deisohexanizer (DIH) unit as will be described in greater detail below.

The FIGURE illustrates an exemplary gasoline producing system in accordance with various embodiments of the present disclosure. As shown therein, exemplary isomerization and deisohexanizer system 10 refines a hydrocarbon feed 12 to create a plurality of products or streams 14, 16, 17, 66, and 70, each of which has a different octane rating, as will be described in greater detail below. In an exemplary embodiment, the feed 12 may primarily include C₅ and C₆ paraffins, and may further include some C₇ paraffins. In general, any suitable paraffin-containing feedstock may be used in the processes of this disclosure.

For example, naphtha feedstocks may be used as the hydrocarbon feed 12 to the isomerization process. Naphtha feedstocks include paraffins, naphthenes, and aromatics, and may include small amounts of olefins, boiling within the gasoline range. Feedstocks which may be utilized include straight-run naphthas, natural gasoline, synthetic naphthas, thermal gasoline, catalytically cracked gasoline, partially reformed naphthas, or raffinates from the extraction of aromatics. The feedstock may be encompassed by a full-range naphtha as defined by boiling points, or from about about 0° to about 230° C. In some embodiments, the feed 12 is a “light” naphtha having an initial boiling point of about 10° to about 65° C. and a final boiling point of about 75° to about 110° C.

Naphtha feedstocks sometimes contain small amounts of sulfur compounds amounting to less than 10 mass parts per million (mppm) on an elemental basis. The naphtha feedstock may be prepared from a contaminated feedstock by a conventional pretreating step such as hydrotreating, hydrorefining, or hydrodesulfurization to convert such contaminants as sulfurous, nitrogenous, and oxygenated compounds to H₂S, NH₃ and H₂O, respectively, which can be separated from hydrocarbons by fractionation. This conversion may employ a catalyst known to the art including an inorganic oxide support and metals selected from Groups VIB(IUPAC 6) and VIII(IUPAC 9-10) of the Periodic Table. Water may act to attenuate catalyst acidity by acting as a base, and sulfur temporarily deactivates the catalyst by platinum poisoning. Feedstock hydrotreating as described hereinabove usually reduces water-generating oxygenates and deactivating sulfur compounds to suitable levels, and other means such as adsorption systems for the removal of sulfur and water from hydrocarbon streams may also be employed, particularly where chlorided alumina catalysts are used. It is within the scope of the present disclosure that this optional pretreating step(s) be included in the present process combination.

The principal components of the hydrocarbon feed 12, in some embodiments, are cyclic and acyclic paraffins having from 4 to 8 carbon atoms per molecule (C₄ to C₈), especially C₅ and C₆, and smaller amounts of aromatic and olefinic hydrocarbons also may be present. Usually, the concentration of C₇ and heavier components is less than about 20, for example less than about 5, mass-percent of the hydrocarbon feed 12, and the concentration of C₄ and lighter components is less than about 20, for example less than about 2, mass-percent of the feedstock. The mass ratio of C₅ to C₆ components in the hydrocarbon feed 12 is from about 1:10 to about 1:1.

Although there are no specific limits to the total content in the feed 12 of cyclic hydrocarbons, the hydrocarbon feed 12 generally contains from about 2 to about 40 mass-percent of cyclics including naphthenes and aromatics. The aromatics contained in the naphtha feedstock, although generally amounting to less than the alkanes and cycloalkanes, may include from about 2 to about 20 mass-percent and more usually about 5 to about 10 mass-percent of the total. Benzene usually includes the principal aromatics constituent of the hydrocarbon feed 12, optionally along with smaller amounts of toluene and higher-boiling aromatics within the boiling ranges described above.

In some embodiments, the feed 12 may optionally have the C₅₋ components thereof substantially removed prior to the introduction thereof to system 10. For example, it will be appreciated that in order to achieve the full octane potential of the gasoline product to be produce, C₅₋ hydrocarbons, such as pentanes, may optionally be removed from this stream in some embodiments. Referring to the FIGURE, depentanizing zone 78a illustrates the optional configuration wherein the feed 12 is depentanized. (In another, alternative embodiment, C₅₋ hydrocarbons depentanizing zone 78b illustrates the alternative, optional configuration wherein the deisohexanizer product 14 is depentanized, and will be described in greater detail below.) With reference first to optional zone 78a, a depentanizer 80a may be provided with an initial feed stream 81, which includes pentanes. The feed stream 81 is fractionated within the depentanizer 80a, such as by conventional distillation, to provide an overhead steam 82a containing C₅₋ hydrocarbons. The bottom stream 12 (referred to above as the feed 12) from the depentanizer column 80a predominantly includes C₆₊ hydrocarbons, which then continues for use as the feed material for system 10. In further alternative embodiments, both zones 78a (described above) and 78b are included in system 10, wherein zone 78a includes a deisopentanizer (80a) and zone 78b includes a depentanizer (as will be described below).

Adverting to the FIGURE, in one embodiment, the feed 12 is received by charge pump 15 and is then fed through line 18 toward an isomerization zone 20. As shown, the output of the charge pump 15 may be combined with make-up hydrogen 22. The make-up hydrogen 22 is combined with line 18 to form a combined feed in line 26. The combined feed in line 26 is then heated by a first indirect heat exchanger 28. Line 30 delivers the output of the first indirect heat exchanger 28 to a second indirect heat exchanger 32 for further heating. The output of the second indirect heat exchanger 32 then flows through line 34 for heating by a third indirect heat exchanger 36. An injection pump 38 adds a chloride source 40, such as perchloroethylene, to the heated output of the third indirect heat exchanger 36 in line 42. The chlorided feed in line 42 is then heated by a charge heater 44 or the like.

As shown, the isomerization zone 20 includes an isomerization unit including a lead isomerization reactor 46 and a lag isomerization reactor 48. While two reactors are shown, in certain embodiments there may be either one or three or more isomerization reactors. Reactors 46 and 48 may be substantially identical, with “lead” and “lag” only referring to their positioning in relation to fluid flow in the system 10. In certain embodiments, the catalyst used in the isomerization zone 20 is distributed equally between the reactors 46 and 48. In other embodiments, there may be differing catalyst distributions. The use of multiple reactors 46 and 48 facilitates a variation in the operating conditions between the two reaction zones to enhance isoparaffin production and improve cyclic hydrocarbon conversion. In this manner, the lead reactor 46 can operate at higher temperature conditions that favor ring opening but performs only a portion of the normal to isoparaffin conversion. The heat exchangers upstream of the lead isomerization reactor 46 facilitate the use of higher temperatures in the lead isomerization reactor 46. Once cyclic hydrocarbon rings have been opened by initial contact with the catalyst, the lag reactor 48 may operate at temperature conditions that are more favorable for isoparaffin equilibrium. In further embodiments (not illustrated), a benzene saturation reactor may additionally be provided. The benzene saturation reactor, if provided, takes the lead position, and operates to convert benzenes into cyclic hexanes.

In some embodiments where, as shown in the FIGURE by stream 70, normal hexane is recycled (the normal hexane being produced as part of product stream 70 from the deisohexanizer, as will be described in greater detail below), the feed 12 and recycle stream 70 are admixed prior to entry into the isomerization zone 20, but if desired, may be separately introduced. In any case, the total feed to the isomerization zone 20 is referred to herein as the isomerization feed (line 30). The recycle may be provided in one or more streams. As discussed in greater detail below, the recycle stream contains linear paraffins, such as normal hexane. The concentration of linear paraffins in the isomerization feed 30 will not only depend upon the concentration of linear paraffins in the feed 12 but also the concentration in the recycle stream 70 and the relative amount of recycle to feed, which may fall within a wide range.

In the isomerization zone 20 the isomerization feed 30 is subjected to isomerization conditions including the presence of isomerization catalyst preferably in the presence of a limited but positive amount of hydrogen as described in U.S. Pat. Nos. 4,804,803 and 5,326,296, both herein incorporated by reference. The isomerization of paraffins is generally considered a reversible first order reaction. Thus, the isomerization reaction effluent will contain a greater concentration of non-linear C₆ paraffins and a lesser concentration of linear C₆ paraffins than does the isomerization feed. The non-linear C₆ paraffins include, for example, methyl pentanes such as 2-methyl pentane and 3-methyl pentane and dimethyl butanes such as 2,2-dimethyl butane and 2,3-dimethyl butane. In some embodiments, the isomerization conditions are sufficient to isomerize at least about 20, for example, from about 30 to about 60, mass-percent of the normal paraffins in the isomerization feed 30. In general, the isomerization conditions achieve at least about 70, for example at least about 75, such as from about 75 to about 97 percent of equilibrium for C₆ paraffins present in the isomerization feed 30. In many instances, the isomerization reaction effluent 54 has a mass ratio of non-linear paraffins to linear paraffins of at least 2:1, preferably between 2.5 to 4:1.

The isomerization catalyst is not critical to the broad aspects of the systems and processes of this disclosure, and any suitable isomerization catalyst may find application. Suitable isomerization catalysts include acidic catalysts using chloride for maintaining the sought acidity and sulfated catalysts. The isomerization catalyst may be amorphous, for example based upon amorphous alumina, or zeolitic. A zeolitic catalyst would still normally contain an amorphous binder. The catalyst may include a sulfated zirconia and platinum as described in U.S. Pat. No. 5,036,035 and European application 0 666 109 A1 or a platinum group metal on chlorided alumina as described in U.S. Pat. Nos. 5,705,730 and 6,214,764. Another suitable catalyst is described in U.S. Pat. No. 5,922,639. U.S. Pat. No. 6,818,589 discloses a catalyst comprising a tungstated support of an oxide or hydroxide of a Group IVB (IUPAC 4) metal, for example zirconium oxide or hydroxide, at least a first component which is a lanthanide element and/or yttrium component, and at least a second component being a platinum-group metal component. These documents are incorporated herein for their teaching as to catalyst compositions, isomerization operating conditions, and associated techniques.

Contacting reactants and catalyst within the isomerization zone 20 may be effected using the catalyst in a fixed-bed system, a moving-bed system, a fluidized-bed system, or in a batch-type operation. A fixed-bed system is employed in exemplary embodiments. The reactants may be contacted with the bed of catalyst particles in upward, downward, or radial-flow fashion. The reactants may be in the liquid phase, a mixed liquid-vapor phase, or a vapor phase when contacted with the catalyst particles, with a primarily liquid-phase operation in some embodiments. As noted above, the isomerization zone 20 may include a single reactor or in two or more separate reactors (46, 48) with suitable means to ensure that the desired isomerization temperature is maintained at the entrance to each zone.

Isomerization conditions in the isomerization zone 20 include reactor temperatures ranging from about 40° to about 250° C. In some embodiments, lower reaction temperatures are provided in order to favor equilibrium mixtures having the highest concentration of high-octane highly branched isoalkanes and to minimize cracking of the feed to lighter hydrocarbons. Temperatures from about 100° to about 200° C. are employed in some embodiments. Reactor operating pressures are generally from about 100 kPa to about 10 MPa absolute, for example from about 0.5 to about 4 MPa absolute. Liquid hourly space velocities may be from about 0.2 to about 25 volumes of isomerizable hydrocarbon feed per hour per volume of catalyst, with about 0.5 to about 15 hr⁻¹ being employed in some embodiments.

As shown the FIGURE, line 50 delivers the output from the charge heater 44 to the lead reactor 46 where isomerization at higher temperatures occurs, producing a hot isomerized stream 52. Isomerized stream 52 is directed to the third indirect heat exchanger 36 where it heats the output of the second indirect heat exchanger 32 carried by line 34. Then, isomerized stream 52 is passed to lag reactor 48 where additional isomerization over the catalysts therein occurs at lower temperatures. As a result of the additional isomerization, a cooler isomerized stream 54 is produced. Isomerized stream 54 is passed through the second indirect heat exchanger 32 and heats the output of the first indirect heat exchanger 28 carried by line 30.

After passing through the second indirect heat exchanger 32, isomerized stream 54 exits the isomerization zone 20 and enters a fractionating column or stabilizer 56. Stabilizer 56 separates an overhead (“offgas”) product 58 typically containing HCl, hydrogen, and light hydrocarbons such as byproduct methane, ethane, propane, and butane gases. Offgas product 58 is scrubbed to remove HCl and then may be routed to a central gas processing plant for removal and recovery of hydrogen, propane, and butane. The residual gas after such processing may become part of the refinery's fuel gas system. In the FIGURE, the stabilizer 56 forms a substantially C₅₊ (or C₆₊ if the C₅ components were previously substantially removed) product removed from a lower end thereof, referred to herein as “bottoms” product 60, which includes liquid isomerate to be fed to a deisohexanizer zone 62. As used herein the term C₅₊ refers to hydrocarbons having five or greater carbon molecules, and C₆₊ refers to hydrocarbons having six or greater carbon molecules. Although not illustrated in the FIGURE, if stream 12 was not previously depentanized, a depentanizing unit may alternatively be included after the stabilizer 56 to depentanize product 60.

In the deisohexanizer zone 62, a deisohexanizer unit 64 deisohexanizes (i.e., separates the branched C₆ components from the linear C₆ components) the bottoms product 60. In some embodiments, the deisohexanizer unit 64 may be a packed or trayed distillation column and may operate with a top pressure of from about 10 to about 500 kPa (gauge) and a bottoms temperature of from about 75° to about 170° C. The deisohexanizer unit 64 produces an over-head product 14, at least one (and optionally two or more) upper side-cut product(s) 16, 17, a mid side-cut product 70, and a heavier, C₇₊ lower end or “bottoms” product 66. The over-head product 14 includes primarily the lightest C₆ isomers and any C₅₋ hydrocarbons that may be present in the stabilizer bottoms product 60. For example, the over-head product 14 may include C₆ isomers, such as 2,2-dimethyl butane and 2,3-dimethyl butane, in addition to any C₅₋ hydrocarbons. Additionally, some heavier branched C₆ isomers may also be present, such as 2-methyl pentane and 3-methyl pentane. In an exemplary embodiment, representing an illustrative example of a system operating upon a depentanized feed, the over-head product 14 contains from about 3% to about 9%, for example about 5% to about 7% pentanes, about 50% to about 65%, for example about 55% to about 60% 2,2-dimethyl butane, about 5% to about 15%, for example about 10% to about 12% 2,3-dimethyl butane, about 14% to about 22%, for example about 16% to about 20% 2-methyl pentane, about 2% to about 4%, for example about 3% 3-methyl pentane, and the remainder other C₆ hydrocarbons (all percentage by mass of the overall product 14). As is known in the art, dimethyl butanes have the highest octane number of the various C₆ isomers, and as such, the over-head product 14 contains the highest octane value of the various products from the deisohexanizer unit 64. In some embodiments, the octane value (RON) of the over-head product 14 may be from about 90 to about 94 or even greater.

As noted above, in order to achieve the full octane potential of the deisohexanizer unit 64 over-head product 14, C₅₋ hydrocarbons, such as pentanes, may optionally be removed from this stream in some embodiments. Again, pentane removal may be accomplished either by depentanizing the feed 12 to the isomerization zone 20 (which was previously described above) or by depentanizing the over-head product 14 from the deisohexanizer unit 64. With reference now to optional zone 78b, a depentanizer 80b may be provided with the over-head product 14, which includes pentanes (the pentanes having not been removed from the initial feed 12 in this embodiment). The product 14 is fractionated within the depentanizer 80b, such as by conventional distillation, to provide an overhead steam 82b containing C₅₋ hydrocarbons. The lower, “bottoms” stream 84b from the depentanizer column 80b predominantly includes C₆₊ hydrocarbons, such as the dimethyl butanes, which may then be used for subsequent high-octane gasoline blending, as described in greater detail below.

The upper side-cut product(s) 16, 17 are withdrawn from the deisohexanizer unit 64 at a point that is above the feed (product 60), but below the over-head product 14. As such, the upper side-cut product(s) 16, 17 include primarily C₆ hydrocarbons that are heavier than the dimethyl butanes withdrawn in the over-head product 14, and that have a lower octane value. For example, in some embodiments, the upper side-cut product(s) 16, 17 include methyl pentanes, such as 2-methyl pentane and 3-methyl pentane, each of which have lower octane ratings than the dimethyl butanes noted above. Of course, some smaller amount of dimethyl butanes may also be present, along with some amount of normal hexanes. In one exemplary embodiment, side-cut product 16 contains about 10% to about 20%, for example about 12% to about 16% 3-methyl pentane, about 30% to about 50%, for example about 37% to about 43% 2-methyl pentane, about 10% to about 20%, for example about 14% to about 18% 2,3-dimethyl butane, and about 20% to about 30%, for example about 22% to about 26% 2,2-dimethyl butane, with the remainder being other C₅ and C₆ hydrocarbons. In embodiments where two or more upper side-cut products are withdrawn, the positioning thereof may be adjusted to withdraw the C₆ isomers in a desired ratio to achieve a desired octane rating of such products, that is, a desired ratio of hexane isomers, with lower cuts containing a greater percentage of lower-octane methyl pentanes and normal hexane. In some embodiments, the octane rating (RON) of the upper side-cut product(s) 16, 17 may be from about 84 to about 91. Where two or more upper side-cut products are present, the octane rating becomes progressively lower as the withdrawal point approaches the feed point.

With this arrangement, two or more C₆-containing streams are produced at different octanes. These two or more different products (14, 16, 17) may thereafter be used as (or as a part of) different grades of gasoline. Alternatively, these two or more different products (14, 16, 17) may be blended into different grades of gasoline, for example they may be blended with each other in various proportions (for example, a portion of stream 14 may be blended with stream 16 in a ratio of about 1:5 or less, such as about 1:10 or less) or they may be blended with other isomerate or reformate products. Blending may be accomplished in a blending system (not shown) which may be provided as part of system 10 or which may be provided separate from system 10. Regardless of the particular embodiment employed, the gasoline product produced thereby may have an octane rating (RON) of about 90 or greater, such as about 92.5 or greater, for example about 95 or greater, and does not include additives such as MTBE, ETBE, or ethanol.

The mid side-cut stream 70 includes normal hexane, 2-methylpentane, 3-methylpentane, the relative amounts of which being dependent on the desired octane levels withdrawn above the mid side-cut stream 70. The exemplary mid side-cut stream 70 may also contain cyclohexane, some dimethyl butanes, and some heavier hydrocarbons. As shown in the FIGURE, the mid side-cut stream 70 passes through the first indirect heat exchanger 28 to heat the combined feed in line 26 upstream of the second indirect heat exchanger 32. The mid side-cut stream 70 then exits the isomerization zone 20 via line 72. As a result of the flows into the first indirect heat exchanger 28, heat is exchanged between the deisohexanizer zone 62 and the isomerization zone 20 upstream of the isomerization unit reactors 46 and 48. After the heat exchange, the mid side-cut stream 70 may be delivered to a cooler 74 to be cooled further, and after cooling, the mid side-cut stream 70 may be fed into the feed 12, as noted above, as a recycle stream. In other embodiments, a portion of the stream 70 may be used as part of a gasoline blend and not recycled.

The lower end or bottoms product 66, as noted above, contains primarily C₇₊ hydrocarbons, and is withdrawn from the deisohexanizer unit 66 through a bottom portion thereof, below the feed stream, for use in other applications. For example, the bottoms product 66 may be used in other hydrocarbon-based fuel blends produced at the same refinery installation.

As such, the presently described embodiments beneficially provide improved methods and systems for producing gasoline at multiple octane grades, and in particular at multiple high octane grades, such as an RON of about 90 or greater. Further, the presently described embodiments provide such methods and systems that do not require the use of imported octane-enhancing materials such as MTBE, ETBE, and/or ethanol.

While at least one exemplary embodiment has been presented in the foregoing detailed description, it should be appreciated that a vast number of variations exist. It should also be appreciated that the exemplary embodiment or embodiments described herein are not intended to limit the scope, applicability, or configuration of the claimed subject matter in any way. Rather, the foregoing detailed description will provide those skilled in the art with a convenient road map for implementing the described embodiment or embodiments. It should be understood that various changes may be made in the processes without departing from the scope defined by the claims, which includes known equivalents and foreseeable equivalents at the time of this disclosure.

What is claimed is:

1. A method for producing a gasoline product comprising the steps of:
isomerizing a first stream comprising normal C₆ hydrocarbons to produce a second stream comprising first and second branched C₆ hydrocarbons;
deisohexanizing the second stream to produce a third stream comprising the first and second branched C₆ hydrocarbons wherein the first branched and the second branched hydrocarbons are present in a first proportion, and a fourth stream comprising the first and second branched hydrocarbons wherein the first branched and the second branched hydrocarbons are present in a second proportion, the first proportion having a relative percentage of first branched hydrocarbons that is greater than a relative percentage of first branched hydrocarbons in the second proportion.

2. The method of claim 1, wherein isomerizing the first stream comprises isomerizing the first stream further comprising C₅ hydrocarbons.

3. The method of claim 2, wherein deisohexanizing the second stream comprises producing the third stream further comprising the C₅ hydrocarbons.

4. The method of claim 3, further comprising depentanizing the third stream.

5. The method of claim 2, further comprising depentanizing or deisopentanizing the first stream, or depentanizing the second stream.

6. The method of claim 1, wherein deisohexanizing the second stream comprises producing the third stream comprising a first product having a first research octane number (RON) and the fourth stream comprising a second product having a second RON that is lower than the first RON.

7. The method of claim 6, further comprising forming a first gasoline product comprising the third stream and forming a second gasoline product comprising the fourth stream and optionally a portion of the third stream, wherein the first gasoline product has a higher RON than the second gasoline product.

8. The method of claim 1, wherein deisohexanizing the second stream comprises producing a fifth stream comprising normal hexane and second branched C₆ hydrocarbons and wherein deisohexanizing the second stream further comprises producing a sixth stream comprising C₇₊ hydrocarbons.

9. The method of claim 1, wherein deisohexanizing the second stream comprises producing the third stream comprising dimethyl butanes and methyl pentanes and further comprises producing the fourth stream comprising dimethyl butanes and methyl pentanes, wherein a proportion of dimethyl butanes in the third stream is greater than a proportion of dimethyl butanes in the fourth stream.

10. The method of claim 9, deisohexanizing the second stream comprises producing the third and fourth streams wherein a proportion of methyl pentanes in the third stream is less than a proportion of methyl pentanes in the fourth stream.

11. A system for producing a gasoline product comprising:
an isomerization unit configured to isomerize normal C₆ hydrocarbons into first and second branched C₆ hydrocarbons;
a deisohexanizing unit, fluidly coupled with the isomerization unit, and configured to separate the first branched C₆ hydrocarbons from the second branched C₆ hydrocarbons, the deisohexanizing unit further configured to produce a first product stream comprising the first branched and the second branched hydrocarbons in a first proportion, and a second product stream comprising the first branched and the second branched hydrocarbons in a second proportion, the first proportion having a relative percentage of first branched hydrocarbons that is greater than a relative percentage of first branched hydrocarbons in the second proportion.

12. The system of claim 11, wherein the isomerization unit comprises first, second, and third isomerization reactors.

13. The system of claim 12, wherein the first isomerization reactor has an operating temperature that is greater than an operating temperature of the second isomerization reactor or an operating temperature of the third isomerization reactor.

14. The system of claim 11, further comprising a depentanizing unit or a deisopentanizing unit fluidly coupled with the isomerization unit to provide a depentanized or deisopentanized feed product to the isomerization unit.

15. The system of claim 11, further comprising a depentanizing unit fluidly coupled with the deisohexanizing unit to produce a depentanized product.

16. The system of claim 11, further comprising a depentanizing unit fluidly coupled with the isomerization unit to provide a depentanized first and second branched C₆ hydrocarbons to the deisohexanizing unit.

17. The system of claim 11, wherein the deisohexanizing unit is further configured to produce a third product stream comprising normal hexanes and second branched C₆ hydrocarbons.

18. The system of claim 17, wherein the deisohexanizing unit is further configured to produce a fourth product stream comprising C₇₊ hydrocarbons.

19. The system of claim 18, wherein the first product stream comprises a first product having a first research octane number (RON) and the second product stream comprises a second product having a second RON that is lower than the first RON.

20. A method for producing a gasoline product comprising the steps of:
isomerizing a first stream comprising pentanes and normal hexane, and C₇₊ hydrocarbons to produce a second stream comprising dimethyl butanes, methyl pentanes, normal hexane, and C₇₊ hydrocarbons;
deisohexanizing the second stream to produce a third stream comprising the dimethyl butanes and methyl pentanes wherein the dimethyl butanes and the methyl pentanes are present in a first proportion, a fourth stream comprising the dimethyl butanes and the methyl pentanes wherein the dimethyl butanes and the methyl pentanes are present in a second proportion, the first proportion having a relative percentage of dimethyl butanes that is greater than a relative percentage of dimethyl butanes in the second proportion, the third stream having a research octane number (RON) that is greater than a RON of the fourth stream, a fifth stream comprising normal hexane and methyl pentanes, and a sixth stream comprising C₇₊ hydrocarbons; and
depentanizing one of the first, second, or third streams, or deisopentanizing the first stream and depentanizing the third stream.
